(19) United States
(12) Patent Application Publication
(10) Pub. No.: US 2025/0228902 A1
(43) Pub. Date: Jul. 17, 2025

(54) COMBINATION DRUG FOR TREATING MALIGNANT TUMOR

(71) Applicant: RIKEN, Wako-shi, Saitama (JP)

(72) Inventors: Hidefumi MUKAI, Wako (JP); Akari KATO, Wako (JP); Shoko NOMURA, Wako (JP)

(73) Assignee: RIKEN, Wako-shi, Saitama (JP)

(21) Appl. No.: 18/849,210

(22) PCT Filed: Mar. 23, 2023
(86) PCT No.: PCT/JP2023/011481

§ 371 Date: Nov. 20, 2024

(30) Foreign Application Priority Data
Mar. 23, 2022 (JP) 2022-047596

(51) Int. Cl. A61K 35/74; A61K 9/127; A61K 31/475; A61K 31/704; A61P 35/00

(52) U.S. Cl. CPC A61K 35/74; A61K 9/127; A61K 31/475; A61K 31/704; A61P 35/00

(57) ABSTRACT

The invention aims to provide a medicament having a superior anti-malignant tumor effect and a method for treating a malignant tumor therewith. In particular, the medicament contains a bacterium and a liposome encapsulating an anti-malignant tumor agent in combination.

### TECHNICAL FIELD

The present invention relates to a medicament for treating malignant tumors, in which a bacterium and a liposome encapsulating an anti-malignant tumor agent are combined.

### BACKGROUND ART

It is known that bacteria (anaerobic bacteria) such as Salmonella selectively survive and grow in tumor tissue when administered intravenously to tumor-bearing living organisms.

Based on the finding that VNP20009, which is an attenuated strain of Salmonella typhimurium, targets tumors and inhibits tumor growth in mice, clinical trials were conducted by intravenous administration thereof to patients with metastatic cancer. Even though the safety of VNP20009 was confirmed, an antitumor effect was not observed (Non Patent Literature 1).

Various studies have been conducted on the treatment of malignant tumors by using attenuated strains of Salmonella. For example, the combined use of an attenuated strain of Salmonella with a tumor-penetrating agent (Patent Literature 1), the use of a recombinant attenuated Gram-negative bacterial strain (Patent Literature 2), the combined use of an attenuated strain of Salmonella with an anti-malignant tumor agent (Non Patent Literatures 2 to 7), and the combined use of an attenuated strain of Salmonella with an antibody (Non Patent Literature 8) have been reported.

In addition, it is known that Listeria monocytogenes, which is one type of Listeria species, Clostridium novyi, which is one type of lostridium species, variants thereof (Clostridium novyi-NT), and the like are used to treat malignant tumors (Non Patent Literature 9).

Anti-malignant tumor agents in which active ingredients are encapsulated in liposomes have been developed as DDS preparations. For example, a preparation in which doxorubicin hydrochloride is encapsulated in liposomes (trade name: Doxil), a preparation in which irinotecan hydrochloride hydrate is encapsulated in liposomes (trade name: Onivyde), a preparation in which vincristine sulfate is encapsulated in liposomes (trade name: Marqibo), a preparation in which daunorubicin citrate is encapsulated in liposomes (trade name: DaunoXome), and the like can be mentioned. It is known that by formulating these active ingredients as DDS preparations by encapsulating them in liposomes, the pharmacokinetics of the active ingredient drug itself change, thus the main pharmacological effect is enhanced and side effects are reduced. Preparations that are characteristically difficult to be recognized as an exogenous substance by the reticuloendothelial system, and that exert an antitumor effect by prolonged blood circulation time and selective extravasation into tumor tissue are also known.

### CITATION LIST

### Patent Literature

### [Patent Literature 1]

WO2015/002969

### [Patent Literature 2]

WO2017/085233

### Non Patent Literature

### [Non Patent Literature 1]

J Clin Oncol. 2002 Jan. 1; 20(1) 142-152

### [Non Patent Literature 2]

Saltzman D., et al., Surgery, 163: 509-514 (2018)

### [Non Patent Literature 3]

Lee C. H., et al., Mol. Ther., 11: 707-716 (2015)

### [Non Patent Literature 4]

Kawaguchi K., et al., Cell Cycle, 17: 2019-2026 (2018)

### [Non Patent Literature 5]

Jia L. J., et al., Int. J. Cancer, 121: 666-674 (2007)

### [Non Patent Literature 6]

Chen J., et al., Theranostics, 7: 2250-2260 (2017)

### [Non Patent Literature 7]

Miyake K., et al., Tissue Cell, 54: 144-149 (2018)

### [Non Patent Literature 8]

Hiroshima Y. et al., PloS One., 10(6): e0120358 (2015)

### [Non Patent Literature 9]

Mai Thi-Quynh Duong, et al., Experimental&Molecular Medicine 51:152 (2019)

### SUMMARY OF INVENTION

### Technical Problem

The present invention aims to provide a novel medicament having a superior anti-malignant tumor effect.

### Solution to Problem

The present inventors have conducted intensive studies with respect to the above-mentioned problems and found that (1) the distribution of liposomes in tumor tissue is improved by allowing bacteria to engraft in the tumor tissue, and (2) the effect of liposomes encapsulating anti-malignant tumor agents is remarkably improved by using them in combination with bacteria such as attenuated strains of Salmonella, that is, a remarkable anti-malignant tumor effect is obtained by combining liposomes encapsulating an anti-malignant tumor agent and bacteria, compared to when they are used alone. Based on such findings, they have conducted further studies and completed the present invention. That is, the present invention provides the following.
[1] A medicament comprising a bacterium and a liposome encapsulating an anti-malignant tumor agent in combination.
[2] The medicament of the above-mentioned [1], wherein the bacterium and the liposome encapsulating an anti-malignant tumor agent are separately formulated and used in combination.
[3] The medicament of the above-mentioned [1] or [2], wherein the bacterium is selected from the group consisting of Salmonella typhimurium, Listeria monocytogenes, Clostridium novyi, and Clostridium novyi-NT.
[4] The medicament of the above-mentioned [1] or [2], wherein the bacterium is Salmonella typhimurium.
[5] The medicament of the above-mentioned [1] or [2], wherein the bacterium is an attenuated strain of Salmonella typhimurium and is selected from the group consisting of VNP20009, A1-R, SHJ2037, SL3261, SL7207, BRD509, and YB1.
[6] The medicament of the above-mentioned [1] or [2], wherein the bacterium is VNP20009.
[7] The medicament of any of the above-mentioned [1] to [6], wherein the anti-malignant tumor agent is selected from the group consisting of doxorubicin and a pharmaceutically acceptable salt thereof, irinotecan and a pharmaceutically acceptable salt thereof, eribulin and a pharmaceutically acceptable salt thereof, gemcitabine and a pharmaceutically acceptable salt thereof, and topotecan and a pharmaceutically acceptable salt thereof.
[8] The medicament of any of the above-mentioned [1] to [6], wherein the anti-malignant tumor agent is doxorubicin or a pharmaceutically acceptable salt thereof.
[9] The medicament of any of the above-mentioned [1] to [6], wherein the anti-malignant tumor agent is irinotecan or a pharmaceutically acceptable salt thereof.
[10] The medicament of any of the above-mentioned [1] to [9], for treating a malignant tumor.
[11] The medicament of any of the above-mentioned [1] to [10], for treating a solid cancer.
[12] The medicament of the above-mentioned [11], wherein the solid cancer is a cancer selected from the group consisting of lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, and skin cancer.
[13] A bacterium for administration simultaneously with or separately from a liposome.
[14] The bacterium of the above-mentioned [13], for use in the treatment of a malignant tumor.
[15] The bacterium of the above-mentioned [14], wherein the malignant tumor is a solid cancer.
[16] The bacterium of the above-mentioned [15], wherein the solid cancer is a cancer selected from the group consisting of lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, and skin cancer.
[17] The bacterium of any of the above-mentioned [13] to [16], which is selected from the group consisting of Salmonella typhimurium, Listeria monocytogenes, Clostridium novyi, and Clostridium novyi-NT.
[18] The bacterium of any of the above-mentioned [13] to [16], which is Salmonella typhimurium.
[19] The bacterium of any of the above-mentioned [13] to [16], which is an attenuated strain of Salmonella typhimurium and is selected from the group consisting of VNP20009, A1-R, SHJ2037, SL3261, SL7207, BRD509, and YB1.
[20] The bacterium of any of the above-mentioned [13] to [16], which is VNP20009.
[21] The bacterium of any of the above-mentioned [13] to [20], wherein the liposome encapsulates an anti-malignant tumor agent.
[22] The bacterium of the above-mentioned [21], wherein the anti-malignant tumor agent is selected from the group consisting of doxorubicin and a pharmaceutically acceptable salt thereof, irinotecan and a pharmaceutically acceptable salt thereof, eribulin and a pharmaceutically acceptable salt thereof, gemcitabine and a pharmaceutically acceptable salt thereof, and topotecan and a pharmaceutically acceptable salt thereof.
[23] The bacterium of the above-mentioned [21], wherein the anti-malignant tumor agent is doxorubicin or a pharmaceutically acceptable salt thereof.
[24] The bacterium of the above-mentioned [21], wherein the anti-malignant tumor agent is irinotecan or a pharmaceutically acceptable salt thereof.
[25] A kit comprising a liposome encapsulating an anti-malignant tumor agent and a preparation containing a bacterium.
[26] The kit of the above-mentioned [25], wherein the bacterium is selected from the group consisting of Salmonella typhimurium, Listeria monocytogenes, Clostridium novyi, and Clostridium novyi-NT.
[27] The kit of the above-mentioned [25], wherein the bacterium is Salmonella typhimurium.
[28] The kit of the above-mentioned [25], wherein the bacterium is an attenuated strain of Salmonella typhimurium and is selected from the group consisting of VNP20009, A1-R, SHJ2037, SL3261, SL7207, BRD509, and YB1.
[29] The kit of the above-mentioned [25], wherein the bacterium is VNP20009.
[30] The kit of any of the above-mentioned [25] to [29], wherein the anti-malignant tumor agent is selected from the group consisting of doxorubicin and a pharmaceutically acceptable salt thereof, irinotecan and a pharmaceutically acceptable salt thereof, eribulin and a pharmaceutically acceptable salt thereof, gemcitabine and a pharmaceutically acceptable salt thereof, and topotecan and a pharmaceutically acceptable salt thereof.
[31] The kit of any of the above-mentioned [25] to [29], wherein the anti-malignant tumor agent is doxorubicin or a pharmaceutically acceptable salt thereof.
[32] The kit of any of the above-mentioned [25] to [29], wherein the anti-malignant tumor agent is irinotecan or a pharmaceutically acceptable salt thereof.
[33] The kit of any of the above-mentioned [25] to [32], for treating a malignant tumor.
[34] The kit of any of the above-mentioned [25] to [32], for treating a solid cancer.
[35] The kit of the above-mentioned [34], wherein the solid cancer is a cancer selected from the group consisting of lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, and skin cancer.
[36] A method for treating a malignant tumor in a subject, comprising a step of administering an effective amount of a combination of a bacterium and a liposome encapsulating an anti-malignant tumor agent to the subject.
[37] The method of the above-mentioned [36], wherein the step of administering to the subject is a step of separately formulating the bacterium and the liposome encapsulating an anti-malignant tumor agent and administering them in combination to the subject.
[38] The method of the above-mentioned [36] or [37], wherein the bacterium is selected from the group consisting of Salmonella typhimurium, Listeria monocytogenes, Clostridium novyi, and Clostridium novyi-NT.
[39] The method of the above-mentioned [36] or [37], wherein the bacterium is Salmonella typhimurium.
[40] The method of the above-mentioned [36] or [37], wherein the bacterium is an attenuated strain of Salmonella typhimurium and is selected from the group consisting of VNP20009, A1-R, SHJ2037, SL3261, SL7207, BRD509, and YB1.
[41] The method of the above-mentioned [36] or [37], wherein the bacterium is VNP20009.
[42] The method of any of the above-mentioned [36] to [41], wherein the anti-malignant tumor agent is selected from the group consisting of doxorubicin and a pharmaceutically acceptable salt thereof, irinotecan and a pharmaceutically acceptable salt thereof, eribulin and a pharmaceutically acceptable salt thereof, gemcitabine and a pharmaceutically acceptable salt thereof, and topotecan and a pharmaceutically acceptable salt thereof.
[43] The method of any of the above-mentioned [36] to [41], wherein the anti-malignant tumor agent is doxorubicin or a pharmaceutically acceptable salt thereof.
[44] The method of any of the above-mentioned [36] to [41], wherein the anti-malignant tumor agent is irinotecan or a pharmaceutically acceptable salt thereof.
[45] The method of any of the above-mentioned [36] to [44], wherein the malignant tumor in the subject is a solid cancer.
[46] The method of the above-mentioned [45], wherein the solid cancer is a cancer selected from the group consisting of lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, and skin cancer.
[47] A combination of a bacterium and a liposome encapsulating an anti-malignant tumor agent, for use as a medicament.
[48] A combination of a bacterium and a liposome encapsulating an anti-malignant tumor agent, for use in treating a malignant tumor.
[49] The combination of the above-mentioned [47] or [48], wherein the bacterium and the liposome encapsulating an anti-malignant tumor agent are separately formulated and used in combination.
[50] The combination of any of the above-mentioned [47] to [49], wherein the bacterium is selected from the group consisting of Salmonella typhimurium, Listeria monocytogenes, Clostridium novyi, and Clostridium novyi-NT.
[51] The combination of any of the above-mentioned [47] to [49], wherein the bacterium is Salmonella typhimurium.
[52] The combination of any of the above-mentioned [47] to [49], wherein the bacterium is an attenuated strain of Salmonella typhimurium and is selected from the group consisting of VNP20009, A1-R, SHJ2037, SL3261, SL7207, BRD509, and YB1.
[53] The combination of any of the above-mentioned [47] to [49], wherein the bacterium is VNP20009.
[54] The combination of any of the above-mentioned [47] to [53], wherein the anti-malignant tumor agent is selected from the group consisting of doxorubicin and a pharmaceutically. acceptable salt thereof, irinotecan and a pharmaceutically acceptable salt thereof, eribulin and a pharmaceutically acceptable salt thereof, gemcitabine and a pharmaceutically acceptable salt thereof, and topotecan and a pharmaceutically acceptable salt thereof.
[55] The combination of any of the above-mentioned [47] to [53], wherein the anti-malignant tumor agent is doxorubicin or a pharmaceutically acceptable salt thereof.
[56] The combination of any of the above-mentioned [47] to [53], wherein the anti-malignant tumor agent is irinotecan or a pharmaceutically acceptable salt thereof.
[57] The combination of any of the above-mentioned [47] to [56], for use in treating a solid cancer.
[58] The combination of the above-mentioned [57], wherein the solid cancer is a cancer selected from the group consisting of lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, and skin cancer.
[59] Use of a combination of a bacterium and a liposome encapsulating an anti-malignant tumor agent, for the production of a medicament for treating a malignant tumor.
[60] The use of the above-mentioned [59], wherein the aforementioned medicament is a medicament in which a bacterium and a liposome encapsulating an anti-malignant tumor agent are separately formulated and used in combination.
[61] The use of the above-mentioned [59] or [60], wherein the bacterium is selected from the group consisting of Salmonella typhimurium, Listeria monocytogenes, Clostridium novyi, and Clostridium novyi-NT.
[62] The use of the above-mentioned [59] or [60], wherein the bacterium is Salmonella typhimurium.
[63] The use of the above-mentioned [59] or [60], wherein the bacterium is an attenuated strain of Salmonella typhimurium and is selected from the group consisting of VNP20009, A1-R, SHJ2037, SL3261, SL7207, BRD509, and YB1.
[64] The use of the above-mentioned [59] or [60], wherein the bacterium is VNP20009.
[65] The use of any of the above-mentioned [59] to [64], wherein the anti-malignant tumor agent is selected from the group consisting of doxorubicin and a pharmaceutically acceptable salt thereof, irinotecan and a pharmaceutically acceptable salt thereof, eribulin and a pharmaceutically acceptable salt thereof, gemcitabine and a pharmaceutically acceptable salt thereof, and topotecan and a pharmaceutically acceptable salt thereof.
[66] The use of any of the above-mentioned [59] to [64], wherein the anti-malignant tumor agent is doxorubicin or a pharmaceutically acceptable salt thereof.
[67] The use of any of the above-mentioned [59] to [64], wherein the anti-malignant tumor agent is irinotecan or a pharmaceutically acceptable salt thereof.
[68] The use of any of the above-mentioned [59] to [67], wherein the aforementioned medicament is a medicament for treating a solid cancer.
[69] The use of the above-mentioned [68], wherein the solid cancer is a cancer selected from the group consisting of lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, and skin cancer.
[70] A medicament for treating a malignant tumor, comprising a bacterium for administration simultaneously with or separately from a liposome encapsulating an anti-malignant tumor agent.
[71] A medicament for treating a malignant tumor, comprising a liposome encapsulating an anti-malignant tumor agent for administration simultaneously with or separately from a bacterium.
[72] An agent for enhancing an anti-malignant tumor action of a bacterium, comprising a liposome encapsulating an anti-malignant tumor agent as an active ingredient.
[73] An agent for enhancing an anti-malignant tumor action of a liposome encapsulating an anti-malignant tumor agent, comprising a bacterium as an active ingredient.
[74] A pharmaceutical composition comprising a combination of a bacterium and a liposome encapsulating an anti-malignant tumor agent.
[75] The pharmaceutical composition of [74], wherein the bacterium and the liposome encapsulating an anti-malignant tumor agent are separately formulated and combined.
[76] The pharmaceutical composition of [74] or [75], wherein the bacterium is an attenuated strain of Salmonella typhimurium and is selected from the group consisting of VNP20009, A1-R, SHJ2037, SL3261, SL7207, BRD509, and YB1.
[77] The pharmaceutical composition of [74] or [75], wherein the bacterium is VNP20009.
[78] The pharmaceutical composition of any of [74] to [77], wherein the anti-malignant tumor agent is doxorubicin or a pharmaceutically acceptable salt thereof.
[79] The pharmaceutical composition of any of [74] to [77], wherein the anti-malignant tumor agent is irinotecan or a pharmaceutically acceptable salt thereof.
[80] The pharmaceutical composition of any of [74] to [79], for treating a malignant tumor.
[81] The pharmaceutical composition of any of [74] to [79], for treating a solid cancer.
[82] The pharmaceutical composition of [81], wherein the solid cancer is a cancer selected from the group consisting of lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, and skin cancer.
[83] The pharmaceutical composition of any of [74] to [82], which is in the form of a kit.
[84] A combination of a bacterium and a liposome encapsulating an anti-malignant tumor agent.
[85] The combination of [84], wherein the bacterium is an attenuated strain of Salmonella typhimurium and is selected from the group consisting of VNP20009, A1-R, SHJ2037, SL3261, SL7207, BRD509, and YB1.
[86] The combination of [84], wherein the bacterium is VNP20009.
[87] The combination of any of [84] to [86], wherein the anti-malignant tumor agent is doxorubicin or a pharmaceutically acceptable salt thereof.
[88] The combination of any of [84] to [86], wherein the anti-malignant tumor agent is irinotecan or a pharmaceutically acceptable salt thereof.

### Advantageous Effects of Invention

The medicament of the present invention characteristically containing a bacterium and a liposome encapsulating an anti-malignant tumor agent in combination has a superior anti-malignant tumor effect.

### BRIEF DESCRIPTION OF DRAWINGS

FIG. 1-1 shows the measurement results of the amount of liposome transferred to U87MG tumor in Experimental Example 1.

FIG. 1-2 shows the observation results of liposome distribution in U87MG tumor (confocal micrographs of tumor tissue sections) in Experimental Example 1.

FIG. 2-1 shows the measurement results of the amount of liposome transferred to A549 tumor in Experimental Example 1.

FIG. 2-2 shows the observation results of liposome distribution in A549 tumor (confocal micrographs of tumor tissue sections) in Experimental Example 1.

FIG. 3-1 shows the measurement results of the amount of liposome transferred to BxPC3 tumor in Experimental Example 1.

FIG. 3-2 shows the observation results of liposome distribution in BxPC3 tumor (confocal micrographs of tumor tissue sections) in Experimental Example 1.

FIG. 4 shows the evaluation results of antitumor effect on A549 tumor and BxPC3 tumor in Experimental Example 2.

FIG. 5 shows the evaluation results of antitumor effect on BxPC3 tumor (comparison test with three administrations) in Experimental Example 3.

FIG. 6 shows the evaluation results of antitumor effect on BxPC3 tumor (effect of single administration) in Experimental Example 3.

FIG. 7 shows the evaluation results of antitumor effect on BxPC3 tumor in Experimental Example 4.

### DESCRIPTION OF EMBODIMENTS

The present invention is described in detail in the following.

The “medicament” in the present invention is not particularly limited as long as it is intended to be used for the diagnosis, treatment, or prevention of the target disease. For example, it encompasses diagnostic agents, therapeutic drugs, prophylactic drugs, pharmaceutical compositions, kits, apparatuses, and use of these, diagnostic methods, treatment methods, prophylactic methods, and the like.

In the medicament of the present invention, a liposome encapsulating an anti-malignant tumor agent and a bacterium are used in combination.

In the medicament of the present invention, the liposome encapsulating an anti-malignant tumor agent, and the bacterium may be simultaneously formulated and contained in the same preparation, or the liposome encapsulating an anti-malignant tumor agent, and the bacterium may be each separately formulated and administered to the same subject simultaneously or with time difference and by the same route or different routes. That is, the medicament of the present invention includes (1) a medicament containing a liposome encapsulating an anti-malignant tumor agent, and a bacterium in one preparation and (2) a medicament in which a liposome encapsulating an anti-malignant tumor agent and a bacterium are separately formulated and used in combination.

The medicament of the present invention can be used by formulating a liposome encapsulating an anti-cancer agent and a bacterium as a single preparation. However, it is preferable to formulate them separately and use them in combination, and they are used for the treatment of malignant tumors, particularly solid cancers.

The bacterium in the present invention can selectively engraft and proliferate in tumor tissue, and can improve the accumulation and distribution of administered liposomes in tumor tissue. That is, when a liposome encapsulating an anti-malignant tumor agent and a bacterium are used in combination in the present invention, the anti-malignant tumor agent encapsulated in the liposomes can be distributed widely within tumor tissue, and the accumulation of the anti-malignant tumor agent in the tumor tissue can be improved, compared to the use of the liposome alone (that is, the concentration of the anti-malignant tumor agent in the tumor tissue can be increased). The bacterium in the present invention is preferably used simultaneously with or separately from the liposome encapsulating an anti-malignant tumor agent, and they are used for the treatment of malignant tumors, particularly solid cancers.

The kit of the present invention is preferably one in which a liposome encapsulating an anti-malignant tumor agent and a bacterium are each formulated and used, and it is used for the treatment of malignant tumors, particularly solid cancers.

In the present invention, the “bacterium” is not particularly limited and includes, for example, Salmonella species (e.g., Salmonella typhimurium), Listeria monocytogenes, Clostridium novyi or variants thereof (Clostridium novyi-NT), etc., and attenuated strains of these, and the like. For the purpose of the present invention, the above-mentioned attenuated strain is preferred. Among others, an attenuated strain of Salmonella species (particularly, Salmonella typhimurium) is more preferred. As the attenuated strain of Salmonella species, VNP20009, A1-R, SHJ2037, SL3261, SL7207, BRD509, YB1, and the like, which are attenuated strains of Salmonella typhimurium, can be specifically mentioned.

In the present invention, the “Salmonella species” refers to gram-negative facultative anaerobic bacilli that belong to a genus (Salmonella) of the Enterobacteriaceae family and live mainly in the gastrointestinal tract of human and animals. Among these, S. typhimurium is one type of Salmonella and is included in the gram-negative facultative anaerobic bacilli/non-typhoid Salmonella species.

The Salmonella used in the present invention is preferably an attenuated strain of Salmonella. For example, S. typhimurium DSLPNG, S. typhimurium A1-R, S. typhimurium VNP20009, S. typhimurium SHJ2037, S. typhimurium SL3261 and the like can be mentioned, and S. typhimurium VNP20009, which is an attenuated strain with purI and msbB mutations and an adenine auxotrophy/LPS mutation, is preferred. In the present invention, “Salmonella species” may be a commercially available product. In the present specification, Salmonella species are sometimes referred to simply as Salmonella.

In the present invention, bacteria can be used alone or by formulating by a known method together with pharmaceutically acceptable additives (e.g., solvent, stabilizer, isotonicity agent, soothing agent, buffering agent, pH adjuster) used generally. For example, bacteria can be contained in an amount of about 10 to about 10¹⁰ cfu, preferably about 10³ to about 10⁹ cfu, more preferably about 10⁵ to about 10⁷ cfu, per 1 ml of an aqueous solution preparation containing isotonicity agent such as sugar, sodium chloride and the like, and the like.

In the present invention, bacteria are preferably used in injections, more preferably in intravenous injections.

In the present specification, injections also include those administered by drip, topical irrigation, catheter, and the like.

The “liposome” of the “liposome encapsulating an anti-malignant tumor agent” to be used in the present invention is not particularly limited, and liposomes known in the field of pharmaceutical preparations can be used.

In the present specification, a liposome is a vesicle of a lipid bilayer membrane having an aqueous interior. Examples of liposome include multilamellar liposomes in which lipid bilayer membranes are stacked in an onion-like manner, and single-lamellar liposomes. The lipid constituting the liposome is generally a phospholipid. Examples of phospholipid include phosphatidyl choline such as lecithin, lysolecithin and the like, acidic phospholipids such as phosphatidyl serine, phosphatidyl glycerol, phosphatidyl inositol, phosphatidic acid and the like, or phospholipids in which the acyl groups of these are substituted with lauroyl group, myristoyl group, oleoyl group, and the like, phosphatidyl ethanolamine, sphingo phospholipids such as sphingo myelin and the like, glycerol glycolipid, cationic lipid, and the like. In addition, the liposome may be combined with a lipid derivative in which a water-soluble polymer such as polyethylene glycol and the like is bound to the lipid. More specifically, for example, hydrogenated soybean phosphatidyl choline (HSPC), 1,2-dimyristoyl-sn-glycerol-3-phosphocholine, 1,2-dipalmitoyl-sn-glycerol-3-phosphocholine (DPPC), 1,2-distearoyl-sn-glycerol-3-phosphocholine (DSPC), 1,2-dilauroyl-sn-glycerol-3-phosphocholine (DLPC), 1,2-dioleoyl-sn-glycerol-3-phosphocholine (DOPC) and the like can be mentioned, and phospholipids to which polyethylene glycol (PEG) is bound, such as N-(carbonyl-methoxypolyethylene glycol 2000)-1,2-distearoyl-sn-glycero-3-phosphoethanolamine and the like can also be used. In addition, cholesterol and the like can also be added. A liposome encapsulating an anti-malignant tumor agent can be produced, for example, by suspending a thin film of purified phospholipid in a solution containing an anti-malignant tumor agent and subjecting the suspension to an ultrasonic treatment or the like. Methods for producing a liposome preparation encapsulating an anti-malignant tumor agent are well known in the art. As regards the production methods, for example, Annals of Oncology, vol. 15, pp. 517-525, 2004; Cancer Science, vol. 95, pp. 608-613, 2004, and the like can be referred to.

The “anti-malignant tumor agent” of the “liposome encapsulating an anti-malignant tumor agent” to be used in the present invention is not particularly limited, and, for example, doxorubicin, irinotecan, eribulin, gemcitabine, topotecan or pharmaceutically acceptable salts of these and the like can be mentioned. Doxorubicin or a pharmaceutically acceptable salt thereof, or irinotecan or a pharmaceutically acceptable salt thereof is preferred. Nucleic acid drugs such as antisense oligonucleotide, siRNA, miRNA, CpGoligo, aptamer and the like can also be utilized as long as they aim to treat malignant tumors.

The “liposome encapsulating an anti-malignant tumor agent” to be used in the present invention is not particularly. limited as long as it is in a form in which an anti-malignant tumor agent is encapsulated as an active ingredient in liposomes. For example, it can be produced by encapsulating. the above-mentioned anti-malignant tumor agent in liposomes by the above-mentioned known methods. In addition, commercially available products such as Doxil (registered trademark), Onivyde (registered trademark), Halaven (registered trade mark) and the like can also be utilized.

In the present invention, liposome preparations and pharmaceutical compositions encapsulating malignant tumor agents may contain pharmaceutically acceptable additives (e.g., solvent, stabilizer, isotonicity agent, soothing agent, buffering agent, pH adjuster) used generally.

In the present invention, liposome preparations and pharmaceutical compositions encapsulating anti-malignant tumor agents are preferably formulated as, for example, injections and the like. In the present invention, when the liposome preparation encapsulating an anti-malignant tumor agent is a liquid preparation such as injection or the like, the preparation may be cryopreserved or stored after removing water by lyophilization. The lyophilized preparation is dissolved again by adding distilled water for injection or the like and then used.

In the medicament of the present invention, the administration method of a liposome encapsulating an anti-malignant tumor agent, and a bacterium is not limited as long as the liposome encapsulating an anti-malignant tumor agent, and the bacterium can reach the affected area or surrounding area thereof. For example, oral or parenteral administration by injection, drip transfusion or the like can be mentioned. Due to the nature of each formulated preparation, parenteral administration is preferred. Specific examples include intravenous administration, intraarterial administration, intramucosal administration, intralymph node administration, and intra-affected tissue administration, and intravenous administration is preferred.

In the medicament of the present invention, the doses of the liposome encapsulating an anti-malignant tumor agent, and the bacterium can be appropriately determined according to the administration route, severity of symptoms, age of patient, extent of side effects and the like.

In the medicament of the present invention, the amount of the liposome encapsulating an anti-malignant tumor agent to be used varies depending on the type of the anti-malignant tumor agent to be used, the type of the liposome, the type of malignant tumor to be treated, the condition of patient and the like. Generally, the doses of commercially available liposomes are used as a reference, and in the case of, for example, an adult (body weight 60 kg), it is 1 mg/m² to 2000 mg/m², preferably 5 mg/m² to 1000 mg/m², more preferably 10 mg/m² to 500 mg/m², per day. The amount of a bacterium to be used varies depending on the type of bacterium to be used, the type of malignant tumor to be the target of treatment, the condition of patient and the like. In the case of, for example, an adult (body weight 60 kg), it is generally about 10 to 10¹⁰ cfu/kg, preferably about 10² to 10⁸ cfu/kg, more preferably about 10³ to 10⁷ cfu/kg, per day. They may be administered at once or in several portions.

The medicament of the present invention can be safely administered to human, mammals other than human (e.g., mouse, rat, rabbit, dog, cat, bovine, horse, monkey, swine, etc.).

The medicament of the present invention is useful as a medicament for treating malignant tumors.

The medicament of the present invention can be particularly effective against solid cancers, among other malignant tumors. More specific examples of the solid cancer include lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, skin cancer and the like, including tumors that have recurred or aggravated after any other treatment, such as chemotherapy, radiation therapy, or the like, for these tumors.

The kit of the present invention may be one in which the liposome encapsulating an anti-malignant tumor agent and the bacterium are each formulated and can be utilized. For example, a product in which a preparation containing a bacterium and a liposome encapsulating an anti-malignant tumor agent are each prepared as a pack, a product in which a preparation containing a bacterium and a liposome encapsulating an anti-malignant tumor agent are prepared such that they can be administered simultaneously or separately via the same tube or separate tubes, and a product in which a liposome encapsulating an anti-malignant tumor agent and a bacterium are formulated in the same pack or the like can be mentioned.

The pharmaceutical composition of the present invention is not particularly limited as long as it contains a liposome encapsulating an anti-malignant tumor agent and a bacterium. For example, it may be formulated to contain each of them or prepared into a kit containing each of them, or each of them may be separately formulated. The pharmaceutical composition of the present invention is applied to pharmaceutical use such as treatment, prevention, and diagnosis of diseases in subjects. Specific examples include those recited in the explanation of the above-mentioned medicaments.

The present invention is explained in more detail in the following based on Examples and Experimental Examples; however, the present invention is not limited to them.

### EXAMPLE

### [Experimental Example 1] Amount Transferred to Tumor/Intratumoral Distribution of Liposome After VNP20009 Administration

Improvement in the amount transferred to various tumors/intratumoral distribution of liposome by the administration of VNP20009 was investigated.

As tumor cells, U87MG cells derived from human glioblastoma, A549 cells derived from human lung cancer, and BxPC3 cells derived from human pancreatic cancer were used.

### 1. Bacteria Culture and Count of Bacteria

The bacteria were cultured using the following bacteria and medium and according to the following procedure, and the obtained bacteria were used as the administration sample for the following test.

### (Bacteria)

Attenuated strain VNP20009 of Salmonella typhimurium (Salmonella enterica serovar typhimurium) (obtained from ATCC)

### (Medium (Both Liquid/Agar))

modified-LB (ingredients: 1% Tryptone, 0.5% Yeast extract, 0.002N CaCl₂, 0.002N MgSO₄; pH: 7.0)

### (Procedure)

(1) 10 μL of bacteria stored frozen at −80° C. in 20% glycerol was added to 4 mL of modified-LB medium.
(2) Cultured overnight at 37° C. with shaking (130 rpm).
(3) The culture medium was diluted 1/20 with modified LB medium and cultured again for 2-3 hr at 37° C. with shaking (130 rpm).
(4) When the optical density (OD600), which indicates turbidity as an index of bacteria quantity, reached 0.5-0.7, 10 mL of the culture medium was recovered.
(5) After centrifugation (8000 g, 3 min), the bacterial pellets obtained were resuspended in 10 mL of saline.
(6) The operation of (5) was repeated.
(7) After centrifugation (8000 g, 3 min), the pellets were resuspended in 10 mL of saline.
(8) The sample was diluted with saline as necessary to prepare an administration sample.

### (Count of Bacteria)

In the following tests, CFU (colony forming unit) was determined by spreading 100 μL of a bacterial suspension diluted as necessary on a modified-LB agar medium, incubating overnight at 37° C., and then counting the number of colonies formed (number of live bacterial cells present in the spread suspension=number of colonies formed).

### 2. Creation of Tumor-Bearing Mouse Model

As tumor cells, U87MG cells derived from human glioblastoma, A549 cells derived from human lung cancer, and BxPC3 cells derived from human pancreatic cancer, were used to prepare tumor-bearing model mice.

Immune-deficient mice (BALB/cSlc nu/nu) with deficient thymic function were used.

80 μL of a suspension of about 4×10⁶ tumor cells was injected subcutaneously into the mice by using a syringe with a 27 G needle. The long and short diameters of the tumor were measured using digital calipers, and the tumor volume was calculated using the formula: (long diameter (mm)×short diameter (mm)×short diameter (mm))/2. When the tumor volume reached about 200 to 400 mm³, it was used in the following test.

### 3. Preparation of Fluorescence-Labeled Liposome

Using the following ingredients and the following preparation method, liposomes (not encapsulating anti-malignant tumor agent) (particle size: about 100 nm) in which the lipid membrane was labeled with the carbocyanine dye DiD were prepared and used as an administration sample for the following tests.

### (Ingredients, Mixing Ratio)

DOPC: 1,2-dioleoyl-sn-glycero-3-phosphocholine (NOF CORPORATION)
Cholesterol (Nacalai Tesque)
mPEG (2000)-DSPE: N-(carbonyl-methoxypolyethyleneglycol 2000)-1,2-distearoyl-sn-glycero-3-phosphorylethanolamine, sodium salt (Laysan Bio, Inc.)
mixing ratio: DOPC/Cholesterol/mPEG (2000)-DSPE=60/40/5 mol % DiD 5 mol % labeling

### (Preparation Method)

1.2 mL of 20 mM HEPES buffer was added to a 15 ml conical tube (Falcon/352095) (Solution 1). 20 mM DOPC, 20 mM Cholesterol, and a solution of 10 mM mPEG (2000)-DSPE in ethanol were added to a 5 mL tube at a molar ratio of 60/40/5. 250 μL of a solution of 1 mM DiD in ethanol was added to the tube, and the total volume was adjusted to 1 mL with isopropyl alcohol (Solution 2). While stirring Solution 1 with a vortex mixer, the entire volume of Solution 2 was added, and after stirring for 30 seconds, 1 mL of PBS(−) was added dropwise. The entire volume was transferred to AmiconUltra-15 (Millipore/MWCO 100,000), ethanol and isopropyl alcohol were removed by ultrafiltration, and the buffer was exchanged, followed by concentration. Finally, the volume was increased to an appropriate volume with PBS(−). The particle size, Polydispersity Index, and electric potential of the liposomes were measured using Zetasizer nano ZSP.

### 4. Administration Method

To the tumor-bearing model mice using U87MG cells, tumor-bearing model mice using A549 cells, and tumor-bearing model mice using BxPC3 cells prepared above were each administered fluorescence-labeled liposomes and VNP20009 by the following administration method.

### (Administration Group)

(i) group administered with fluorescence-labeled liposomes but not with VNP20009 (control group, hereinafter referred to as the “VNP20009(−) group”)
(ii) group administered with VNP20009 and administered with fluorescence-labeled liposomes immediately thereafter (hereinafter referred to as the “VNP20009(+)DAY0 group”)
(iii) group administered with VNP20009 and administered with fluorescence-labeled liposomes 3 days later (hereinafter referred to as the “VNP20009(+)DAY3 group”)

### (Administration Method, Evaluation Schedule)

### (i) VNP20009(−) Group

Fluorescence-labeled liposomes (about 1 μmol/mouse in terms of lipid amount) were administered into the tail vein of tumor-bearing model mice under isoflurane anesthesia by using a syringe with a 29 G needle. One day after administration of the fluorescence-labeled liposomes, tumor tissues were collected from the tumor-bearing model mice.

### (ii) VNP20009(+)DAY0 Group

VNP20009 (2 to 4×10⁶ CFU/mouse) was administered into the tail vein of tumor-bearing model mice under isoflurane anesthesia by using a syringe with a 29 G needle, and fluorescence-labeled liposomes (about 1 mol/mouse in terms of lipid amount) were administered into the tail vein immediately thereafter (within 1-2 min) using a syringe with a 29 G needle. One day after administration of the fluorescence-labeled liposomes, tumor tissues were collected from the tumor-bearing model mice.

(iii) VNP20009(+)DAY3 Group

VNP20009 (2 to 4×10⁶ CFU/mouse) was administered into the tail vein of tumor-bearing model mice under isoflurane anesthesia by using a syringe with a 29 G needle, and fluorescence-labeled liposomes (about 1 μmol/mouse in terms of lipid amount) were administered into the tail vein 3 days later using a syringe with a 29 G needle. One day after administration of the fluorescence-labeled liposomes, tumor tissues were collected from the tumor-bearing model mice.

### 5. Evaluation Method

### 5-1. Measurement of Amount of Fluorescence-Labeled Liposomes Transferred to Tumor

(1) Tumor tissue collected from mice was diluted 10-fold with saline and crushed to make a tumor tissue suspension.
(2) 500 μL of the tumor tissue suspension was mixed with 500 μL of 2% SDS (sodium dodecyl sulfate) solution to dissolve the tissue.
(3) After centrifugation (4° C., 10000 g, 10 min), 600 μL of the supernatant was collected and used as a measurement sample.
(4) A similar procedure was performed using tumor tissue collected from tumor-bearing model mice free of fluorescence-labeled liposome administration and an arbitrary amount of fluorescence-labeled liposomes to prepare calibration curve samples of fluorescence intensity.
(5) The fluorescence intensity of the calibration curve sample and measurement sample was measured, and the amount of the fluorescence-labeled liposomes in the tumor tissue was calculated.

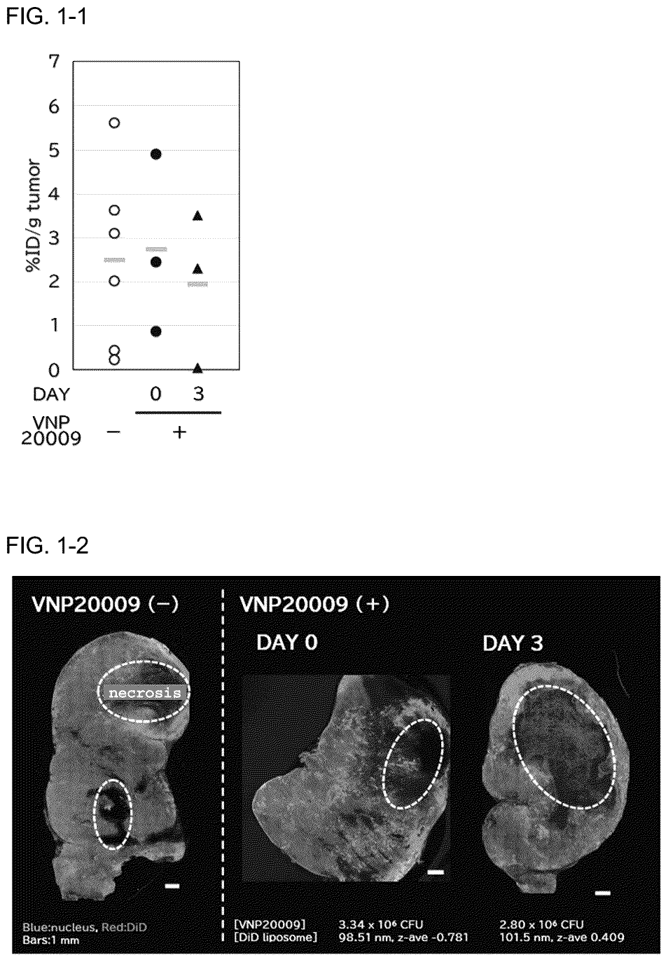

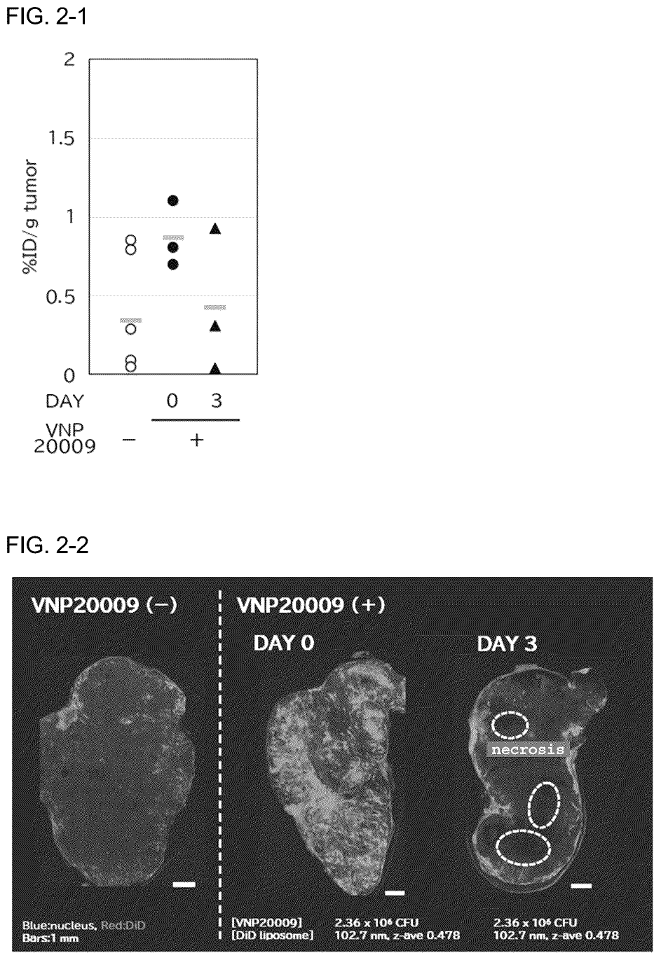

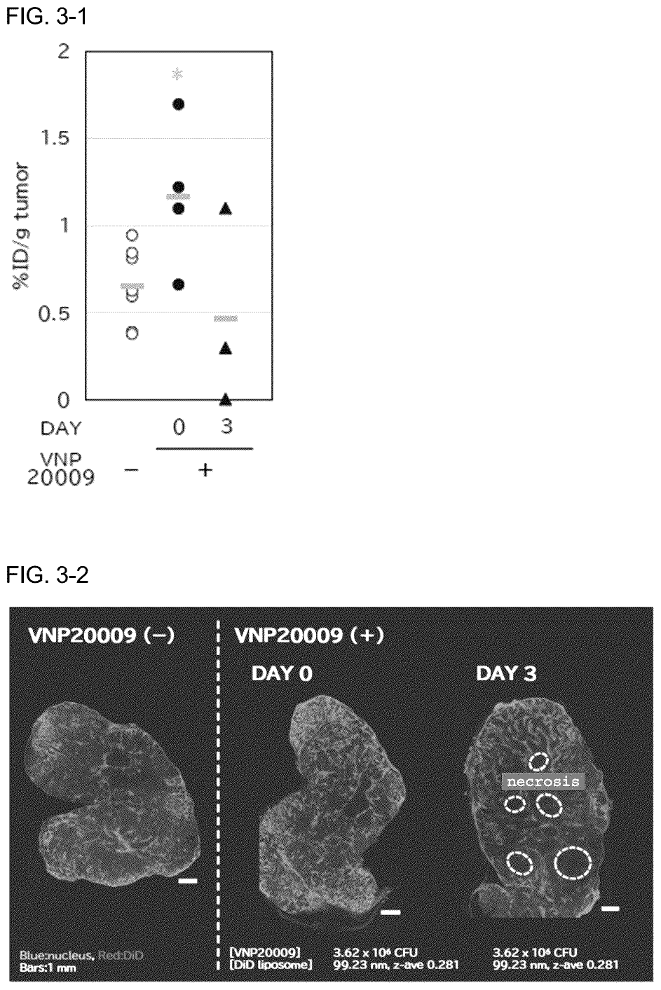

The measurement results of the amount of the fluorescence-labeled liposomes transferred to U87MG tumor are shown in FIG. 1-1, the measurement results of the amount transferred to A549 tumor are shown in FIG. 2-1, and the measurement results of the amount transferred to BxPC3 tumor are shown in FIG. 3-1.

### 5-2. Observation of Intratumoral Distribution of Fluorescence-Labeled Liposomes

(1) Tumor tissues collected from mice were placed in 4% PFA (paraformaldehyde) and immersed and fixed overnight at 4° C.
(2) The fixed tumor tissue was sliced into a thickness of 300 μm.
(3) Cell nucleus was stained using Hoechst 33342 (shown in blue in the image).
(4) Cell nucleus (shown in blue) and liposome fluorescence (shown in red) were observed using a confocal microscope.

The observation results of U87MG intratumoral distribution of fluorescence-labeled liposomes are shown in FIG. 1-2, the observation results of A549 intratumoral distribution are shown in FIG. 2-2, and the observation results of BxPC3 intratumoral distribution are shown in FIG. 3-2.

FIG. 1-2, FIG. 2-2, and FIG. 3-2 are originally color photographs, where the light gray areas are originally red and the dark gray areas are originally blue.

As a result of the observation of U87MG intratumoral distribution of fluorescence-labeled liposomes (FIG. 1-2), liposome fluorescence (shown in red) was distributed only in the outer portion of the tissue in the VNP20009(−) group (FIG. 1-2, left photograph). In the VNP20009(+)DAY0 group (FIG. 1-2, center photograph) and VNP20009(+)DAY3 group (FIG. 1-2, right photograph), the fluorescence of the liposomes (shown in red) also spread within the tissue, and it was observed that the intratumoral distribution area of the liposomes was enlarged. In addition, in the VNP20009(+)DAY3 group (FIG. 1-2, right photograph), the necrotic area (area surrounded by dotted line) was enlarged.

From the measurement results of the amount of fluorescence-labeled liposomes transferred to A549 tumor (FIG. 2-1), the VNP20009(+)DAY0 group showed a remarkably increased amount of liposomes transferred to tumor, compared to the VNP20009(−) group.

From the observation results of A549 intratumoral distribution of fluorescence-labeled liposomes (FIG. 2-2), liposome fluorescence. (shown in red) was distributed only at the outer portion of the tissue in the VNP20009(−) group (FIG. 2-2, left photograph), the fluorescence of the liposomes (shown in red) also spread within the tissue in the VNP20009(+)DAY0 group (FIG. 2-2, center photograph), and it was found that the distribution area was enlarged. In the VNP20009(+)DAY3 group (FIG. 2-2, right photograph), the necrotic areas (area surrounded by dotted line) were scattered.

From the measurement results of the amount of fluorescence-labeled liposomes transferred to BxPC3 tumor (FIG. 3-1), the VNP20009(+)DAY0 group showed a significantly increased amount of liposomes transferred to tumor, compared to the VNP20009(−) group (*p<0.05, non-repeated ANOVA followed by SNK test).

From the observation results of BxPC3 intratumoral distribution of fluorescence-labeled liposomes (FIG. 3-2), some areas where the distribution of liposome fluorescence (shown in red) was low inside the tissue were scattered in the VNP20009(−) group (FIG. 3-2, left photograph), the fluorescence of the liposomes (shown in red) spread within the entire area in the tissue in the VNP20009(+)DAY0 group (FIG. 3-2, center photograph) and VNP20009(+)DAY3 group (FIG. 3-2, right photograph), and it was found that the distribution area was enlarged. In the VNP20009(+)DAY3 group (FIG. 3-2, right photograph), the necrotic areas (area surrounded by dotted line) were scattered.

From the above-mentioned results, it was confirmed that administration of Salmonella (VNP20009) promotes the accumulation of liposomes in tumor tissue, that combined administration of Salmonella (VNP20009) and liposomes (particularly administration of liposomes immediately after (substantially simultaneously with) Salmonella (VNP20009) administration) increases the amount of liposomes transferred to tumor tissue, and that administration of liposomes after (3 days after) Salmonella (VNP20009) administration can increase the distribution of liposomes in tumor tissue.

From the above-mentioned results, moreover, it was confirmed that the distribution area of liposomes in tumor tissue can be expanded by administering Salmonella (VNP20009) in combination with liposomes (particularly by administering liposomes immediately after administration of Salmonella (VNP20009) or by administering liposomes after some time from the administration of Salmonella (VNP20009)).

### [Experimental Example 2] Antitumor Effect of Combined Use of Liposome Encapsulating an Anti-Malignant Tumor Agent and VNP20009

A549 tumor and BxPC3 tumor with increased amount of liposome transferred to tissue by the combined administration with VNP20009 in Experimental Example 1 were evaluated for the antitumor effect by the combined use of VNP20009 and liposomes encapsulating an anti-malignant tumor agent.

### 1. Bacteria Culture and Count of Bacteria

Bacteria (VNP20009 (obtained from ATCC)) were cultured in the same manner as in Experimental Example 1, and the obtained bacteria were used as an administration sample for the following tests.

### 2. Creation of Tumor-Bearing Mouse Model

In the same manner as in Experimental Example 1, tumor-bearing model mice were prepared using human lung cancer-derived A549 cells and human pancreatic cancer-derived BxPC3 cells as tumor cells.

### 3. Liposome Encapsulating Anti-Malignant Tumor Agent

As liposome encapsulating an anti-malignant tumor agent, doxorubicin-encapsulated liposomes (“Doxil (registered trademark) Injection 20 mg”) were used in the following tests.

### 4. Administration Method

To the tumor-bearing mice using A549 cells and the tumor-bearing mice using BxPC3 cells prepared above were administered (a) a combination of doxorubicin-encapsulated liposomes and VNP20009 (combined use administration group), (b) doxorubicin-encapsulated liposomes alone (liposome administration group), and (c) VNP20009 alone (VNP20009 administration group) by the following administration methods. As (d) control group, a group without administration was established.

### (Administration Method)

### (a) Combined Use Administration Group

VNP20009 (about 5×10⁵ CFU/mouse) was administered into the tail vein of tumor-bearing model mice under isoflurane anesthesia by using a syringe with a 29 G needle, and doxorubicin-encapsulated liposomes (5 mg/kg body weight in terms of doxorubicin amount) were administered into the tail vein immediately thereafter (within 1-2 min) using a syringe with a 29 G needle. 7 days and 14 days after administration, VNP20009 and doxorubicin-encapsulated liposomes were. administered by the same method.

### (b) Liposome Administration Group

Under isoflurane anesthesia, doxorubicin-encapsulated liposomes (5 mg/kg body weight in doxorubicin amount) were administered into the tail vein of tumor-bearing model mice by using a syringe with a 29 G needle. 7 days and 14 days after administration, doxorubicin-encapsulated liposomes were administered by the same method.

### (c) VNP20009 Administration Group

Under isoflurane anesthesia, VNP20009 (about 5×10⁵ CFU/mouse) was administered into the tail vein of tumor-bearing model mice by using a syringe with a 29 G needle. 7 days and 14 days after administration, VNP20009 was administered by the same method.

### 5. Evaluation Method

From the first day of administration until the 18th day, every day or once every two days, the long and short diameters of the tumor were measured using digital calipers, and the tumor volume was calculated using the formula: (long diameter (mm)×short diameter (mm)×short diameter (mm))/2.

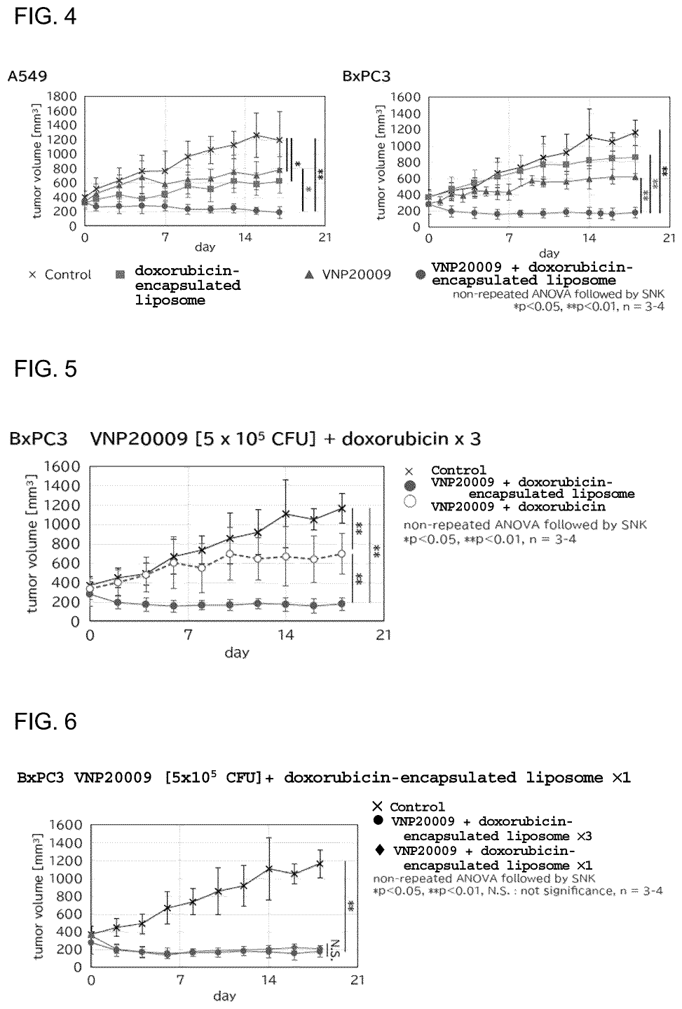

The results are shown in FIG. 4.

From the results of FIG. 4, it was confirmed that the combined administration of doxorubicin-encapsulated liposomes and VNP20009 (“VNP20009+doxorubicin-encapsulated liposome” in FIG. 4) provides a stronger antitumor effect (effect of suppressing an increase in tumor volume) than the sole administration of each (“VNP20009” or “doxorubicin-encapsulated liposome” in FIG. 4). In addition, it was shown that the combined administration of doxorubicin-encapsulated liposomes and VNP20009 (“VNP20009+doxorubicin-encapsulated liposome” in FIG. 4) affords an antitumor effect even at a dose at which VNP20009 alone is less effective. It was considered that these effects afforded by the combined administration of doxorubicin-encapsulated liposomes and VNP20009 could not be predicted from the effects of each administered alone.

Therefore, the combined use of a liposome encapsulating an anti-malignant tumor agent and a bacterium such as VNP20009 or the like is expected to have a strong antitumor action at a low dose, suggesting that it may be an effective treatment method even for cancer types that are generally difficult to treat.

### [Experimental Example 3] Antitumor Effect by Combined Use of Liposome Encapsulating Anti-Malignant Tumor Agent and VNP20009 (Comparison Test of Combined Use of Anti-Malignant Tumor Agent and VNP20009)

The antitumor effect of combined use of liposomes encapsulating an anti-malignant tumor agent and VNP20009 against BxPC3 tumor was evaluated.

For comparison, the anti-malignant tumor effect of combined use of an anti-malignant tumor agent and VNP20009 was examined.

### 1. Bacteria Culture and Count of Bacteria

Bacteria (VNP20009 (obtained from ATCC)) were cultured in the same manner as in Experimental Example 1, and the obtained bacteria were used as an administration sample for the following tests.

### 2. Creation of Tumor-Bearing Mouse Model

In the same manner as in Experimental Example 1, tumor-bearing model mice were prepared using human pancreatic cancer-derived BxPC3 cells as tumor cells.

### 3. Liposome Encapsulating Anti-Malignant Tumor Agent, and Anti-Malignant Tumor Agent

As liposome encapsulating an anti-malignant tumor agent, doxorubicin-encapsulated liposomes (“Doxil (registered trademark) Injection 20 mg”) were used in the following tests.

As the anti-malignant tumor agent, doxorubicin hydrochloride (FUJIFILM Wako Pure Chemical Corporation) was used in the following tests.

### 4. Administration Method

To the tumor-bearing mice using BxPC3 cells prepared above were administered (a) a combination of doxorubicin-encapsulated liposomes and VNP20009 (liposome combined use administration group), and (b) a combination of doxorubicin hydrochloride and VNP20009 (doxorubicin combined use administration group) by the following administration methods. As (c) control group, a group without administration was established.

The combined administration of (a) doxorubicin-encapsulated liposomes and VNP20009 was investigated by 3 times of administration (administered on day 0, day 7, and day 14) and also by single administration (administered on day 0 alone).

### (Administration Method)

(1) 3 times of Administration (Administered on Day 0, Day 7, and Day 14)

### (a) Liposome Combined Use Administration Group

VNP20009 (about 5×10⁵ CFU/mouse) was administered into the tail vein of tumor-bearing model mice under isoflurane anesthesia by using a syringe with a 29 G needle, and doxorubicin-encapsulated liposomes (5 mg/kg body weight in terms of doxorubicin amount) were administered into the tail vein immediately thereafter (within 1-2 min) using a syringe with a 29 G needle. 7 days and 14 days after administration, VNP20009 and doxorubicin-encapsulated liposomes were administered by the same method.

### (b) Doxorubicin Combined Use Administration Group

VNP20009 (about 5×10⁵ CFU/mouse) was administered into the tail vein of tumor-bearing model mice under isoflurane anesthesia by using a syringe with a 29 G. needle, and doxorubicin hydrochloride (5 mg/kg body weight) was administered into the tail vein immediately thereafter (within 1-2 min) using a syringe with a 29 G needle. 7 days and 14 days after administration, doxorubicin hydrochloride was administered by the same method.

### (2) Administration Only Once (Administered on Day 0 Alone)

### (a) Liposome Combined Use Administration Group

VNP20009 (about 5×10⁵ CFU/mouse) was administered into the tail vein of tumor-bearing model mice under isoflurane anesthesia by using a syringe with a 29 G needle, and doxorubicin-encapsulated liposomes (5 mg/kg body weight in terms of doxorubicin amount) were administered into the tail vein immediately thereafter (within 1-2 min) using a syringe with a 29 G needle.

### 5. Evaluation Method

From the first day of administration until the 18th day, once every two days, the long and short diameters of the tumor were measured using digital calipers, and the tumor volume was calculated using the formula: (long diameter (mm)×short diameter (mm)×short diameter (mm))/2.

FIG. 5 shows the results of 3 times of administration (administered on day 0, day 7, and day 14) in (a) liposome combined use administration group (“VNP20009+doxorubicin-encapsulated liposome” in FIG. 5), (b) doxorubicin combined use administration group (“VNP20009+doxorubicin” in FIG. 5), and (c) control group (“Control” in FIG. 5).

FIG. 6 shows the results of administration one time (administered on day 0 alone) in (a) liposome combined use administration group (“VNP20009+doxorubicin-encapsulated liposome×1” in FIG. 6). FIG. 6 also shows the results of 3 times of administration (administered on day 0, day 7, and day 14) shown in FIG. 5 in (a) liposome combined use administration group (“VNP20009+doxorubicin-encapsulated liposome×3” in FIG. 6) and (c) control group (“Control” in FIG. 6).

From the results of FIG. 5, it was confirmed that the combined administration of doxorubicin-encapsulated liposomes and VNP20009 provides a stronger antitumor effect (effect of suppressing an increase in tumor volume) than the combined administration of doxorubicin hydrochloride and VNP20009.

From the results of FIG. 6, it was confirmed that the combined administration of doxorubicin-encapsulated liposomes and VNP20009, which were administered only once (on day 0 alone), provided a strong antitumor effect (effect of suppressing an increase in tumor volume) equivalent to that achieved by administering the combination three times (on days 0, 7, and 14).

### [Experimental Example 4] Antitumor Effect by Combined Use of Liposome Encapsulating Anti-Malignant Tumor Agent and VNP20009

The antitumor effect of combined use of VNP20009 and a liposome encapsulating an anti-malignant tumor agent against BxPC3 tumor was evaluated.

### 1. Bacteria Culture and Count of Bacteria

Bacteria (VNP20009 (obtained from ATCC)) were cultured in the same manner as in Experimental Example 1, and the obtained bacteria were used as an administration sample for the following tests.

### 2. Creation of Tumor-Bearing Mouse Model

In the same manner as in Experimental Example 1, tumor-bearing model mice were prepared using human pancreatic cancer-derived BxPC3 cells as tumor cells. When the tumor volume reached about 200 mm³, they were used in the following test.

### 3. Liposome Encapsulating an Anti-Malignant Tumor Agent

As liposome encapsulating an anti-malignant tumor agent, irinotecan-encapsulated liposomes (“Onivyde (registered trademark) drip intravenous injection 43 mg”) were used in the following tests.

### 4. Administration Method

To the tumor-bearing mice using BxPC3 cells prepared above were administered (a) a combination of irinotecan-encapsulated liposomes and VNP20009 (combined use administration group), and (b) irinotecan-encapsulated liposomes alone (liposome administration group) by the following administration methods.

### (Administration Method)

### (a) Combined Use Administration Group

VNP20009 (2.8×10⁵ CFU/mouse) was administered into the tail vein of tumor-bearing model mice under isoflurane anesthesia by using a syringe with a 29 G needle, and irinotecan-encapsulated liposomes (20 mg/kg body weight in terms of irinotecan amount) were administered into the tail vein immediately thereafter (within 1-2 min) using a syringe with a 29 G needle (n=3).

### (b) Liposome Administration Group

Irinotecan-encapsulated liposomes (20 mg/kg body weight in terms of irinotecan amount) were administered into the tail vein of tumor-bearing model mice under isoflurane anesthesia by using a syringe with a 29 G needle (n=3).

### 5. Evaluation Method

On the first day of administration (day 0) and the 2nd day, the 6th day, the 9th day, the 12th day, and the 15th day from administration, the long and short diameters of the tumor were measured using digital calipers, and the tumor volume was calculated using the formula: (long diameter (mm)×short diameter (mm)×short diameter (mm))/2. Tumor growth rate (%) was calculated by taking the value on the day of administration (day 0) as 100.

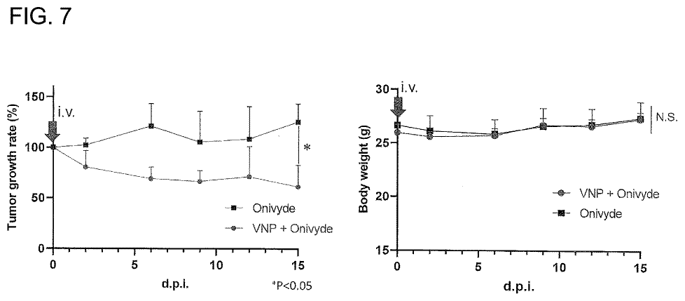

The results are shown in FIG. 7.

From the results of FIG. 7, it was confirmed that the combined administration of irinotecan-encapsulated liposomes and VNP20009 (“VNP+Onivyde (trade name)” in FIG. 7) provides a stronger antitumor effect than the sole administration of irinotecan-encapsulated liposomes (“Onivyde (trade name)” in FIG. 7) (FIG. 7, left). A decrease in the body weight was not observed in any groups (FIG. 7, right).

Therefore, the combined use of a liposome encapsulating an anti-malignant tumor agent and a bacterium such as VNP20009 or the like is expected to have a strong antitumor action, suggesting that it may be an effective treatment method even for cancer types that are generally difficult to treat.

### Formulation Example 1

The following (1) and (2) are mixed to produce an injection for anti-malignant tumor containing (1) and (2).
(1) Bacterium (VNP20009) suspension obtained by the method of Experimental Example 1, 10 mL (containing 2×10⁶ CFU)
(2) Doxorubicin-encapsulated liposomes (Doxil (registered trademark) Injection 20 mg), 10 mL

### INDUSTRIAL APPLICABILITY

According to the present invention, a novel medicament having a superior anti-malignant tumor effect can be provided.

This application is based on patent application No. 2022-047596 filed in Japan, the contents of which are encompassed in full herein.

1.-9. (canceled)

10. A method for treating a malignant tumor in a subject, comprising a step of administering an effective amount of a combination of a bacterium and a liposome encapsulating an anti-malignant tumor agent to the subject.

11. The method according to claim 10, wherein the step of administering to the subject is a step of separately formulating the bacterium and the liposome encapsulating an anti-malignant tumor agent and administering them in combination to the subject.

12. The method according to claim 10, wherein the bacterium is an attenuated strain of Salmonella typhimurium and is selected from the group consisting of VNP20009, A1-R, SHJ2037, SL3261, SL7207, BRD509, and YB1.

13. The method according to claim 10, wherein the bacterium is VNP20009.

14. The method according claim 10, wherein the anti-malignant tumor agent is doxorubicin or a pharmaceutically acceptable salt thereof.

15. The method according to claim 10, wherein the anti-malignant tumor agent is irinotecan or a pharmaceutically acceptable salt thereof.

16. The method according to claim 10, wherein the malignant tumor in the subject is a solid cancer.

17. The method according to claim 16, wherein the solid cancer is a cancer selected from the group consisting of lung cancer, pancreatic cancer, glioblastoma, ovarian cancer, Kaposi's sarcoma, multiple myeloma, breast cancer, osteosarcoma, esophageal cancer, liver cancer, gastric cancer, pancreatic cancer, colorectal cancer, rectal cancer, colon cancer, ureter tumor, brain tumor, gall bladder cancer, bile duct cancer, biliary tract cancer, renal cancer, bladder cancer, cervical cancer, prostate cancer, thyroid cancer, orchis tumor, maxilla cancer, tongue cancer, lip cancer, oral cavity cancer, pharyngeal cancer, laryngeal cancer, myosarcoma, and skin cancer.

18.-45. (canceled)

46. The method according to claim 11, wherein the bacterium is an attenuated strain of Salmonella typhimurium and is selected from the group consisting of VNP20009, A1-R, SHJ2037, SL3261, SL7207, BRD509, and YB1.

47. The method according to claim 11, wherein the bacterium is VNP20009.

48. The method according to claim 11, wherein the anti-malignant tumor agent is doxorubicin or a pharmaceutically acceptable salt thereof.

49. The method according to claim 12, wherein the anti-malignant tumor agent is doxorubicin or a pharmaceutically acceptable salt thereof.

50. The method according to claim 13, wherein the anti-malignant tumor agent is doxorubicin or a pharmaceutically acceptable salt thereof.

51. The method according to claim 11, wherein the anti-malignant tumor agent is irinotecan or a pharmaceutically acceptable salt thereof.

52. The method according to claim 12, wherein the anti-malignant tumor agent is irinotecan or a pharmaceutically acceptable salt thereof.

53. The method according to claim 13, wherein the anti-malignant tumor agent is irinotecan or a pharmaceutically acceptable salt thereof.
